(19) United States
(12) Patent Application Publication
(10) Pub. No.: US 2024/0363200 A1
(43) Pub. Date: Oct. 31, 2024

(54) REAL-TIME VIRUS AND DAMAGING AGENT DETECTION

(71) Applicant: INTELLISAFE LLC, Denver, CO (US)

(72) Inventor: Trevor CHANDLER, Thornton, CO (US)

(21) Appl. No.: 18/685,779

(22) PCT Filed: Aug. 29, 2022
(86) PCT No.: PCT/US2022/041874

§ 371 Date: Feb. 22, 2024

Related U.S. Application Data
(60) Provisional application No. 63/238,644, filed on Aug. 30, 2021.

(51) Int. Cl. G16B 35/20; G01N 33/543; G01N 33/569; G16B 40/20

(52) U.S. Cl. CPC G16B 35/20; G01N 33/54386; G01N 33/56983; G16B 40/20; G01N 2500/20

(57) ABSTRACT

The present disclosure describes systems and methods for real-time virus and damaging agent detection, and more specifically, for capturing images of viruses and other damaging agents in real-time and utilizing such information to detect the presence of viruses and other damaging agents. In operation a sampling device may generate a digital pattern from a molecular sample. A computing device may, using the virus and damaging agent detection machine-learning model, analyze the digital pattern to a 3D model of a particular damaging agent. Based on the analysis exceeding an identification threshold, the computing device may identify the presence of the particular damaging agent within the molecular sample. The computing device may send an alert to a user device indicative of the presence of the particular damaging agent within the molecular sample.

### CROSS-REFERENCE TO RELATED APPLICATIONS

This application claims the benefit of priority of U.S. Provisional Patent Application No. 63/238,644, filed Aug. 30, 2021, entitled “Real-Time Virus and Damaging Agent Detection,” which is hereby incorporated by reference herein in its entirety.

### FIELD

Examples described herein generally relate to real-time virus and damaging agent detection, and more specifically, to capturing images of viruses and other damaging agents in real-time and utilizing such information to detect the presence of viruses and other damaging agents.

### BACKGROUND

Artificial intelligence (AI) is the simulation of human intelligence processes by machines, particularly in computer systems. Machine-learning is an application of AI that provides computer systems the ability to automatically learn and improve from experience without being explicitly programmed. While in the beginning the integration and application of AI and machine-learning was limited, after decades of development, AI and machine-learning currently permeate numerous fields and have helped advance and develop various industries, from finance, business, and education, to agriculture, telecommunications, and transportation.

The healthcare industry has also leveraged the benefits of AI in both research-and clinical-based practice. For example, AI and machine-learning aids in diagnosis processes, treatment protocol development, drug synthesis, personalized medicine, patient monitoring, and the like. However, as the spread of new and existing viruses, bacteria, and other contaminants becomes a cause of global concern, the scientific and medical communities have struggled to fully take advantage of advancements in technology and apply them to diagnostics and tracking, in order to limit the spread of such viruses and disease.

### SUMMARY

The present application includes a method for determining the presence of a damaging agent. The method includes receiving, from a sampling device, a digital pattern of a molecular sample; analyzing, at a computing device comprising a virus and damaging agent machine-learning model and communicatively coupled to the sampling device, the received digital pattern of the molecular sample; and based on the analyzing, identifying, using the virus and damaging agent machine-learning model, a particular damaging agent within the molecular sample, wherein the identifying is further based on the digital pattern exceeding an identification threshold.

Additionally, a system is disclosed. The system includes a sampling device configured to generate a digital pattern of a molecular sample and a computing device including a virus and damaging agent detection machine-learning model and communicatively coupled to the sampling device. The computing device is configured to receive from the sampling device the digital pattern of the molecular sample, analyze the received digital pattern of the molecular sample, and based on the analysis, identify a particular damaging agent within the molecular sample, where the identifying is further based on the digital pattern exceeding an identification threshold.

A method for training a virus and damaging agent detection machine-learning model used for detecting a damaging agent from a digital pattern of a molecular sample is disclosed. The method includes generating a three dimensional model of a particular damaging agent in a particular environment, utilizing the 3D model to generate a plurality of output images, where the plurality of output images are captured at one or more of different rotations of the 3D model, varying brightness levels, or varying magnification levels, and training, using the plurality of output images, the virus and damaging agent detection machine-learning model to detect the damaging agent from the digital pattern of the molecular sample.

### BRIEF DESCRIPTION OF THE DRAWINGS

Reference is now made to the following descriptions taken in conjunction with the accompanying drawings, in which:

FIG. 1A is a schematic illustration of a system for real-time virus and damaging agent detection, in accordance with examples described herein;

FIG. 1B is a schematic illustration of a system for real-time virus and damaging agent detection, in accordance with examples described herein;

FIG. 2A is an example protective facemask sampling device, in accordance with examples described herein;

FIG. 2B is an example protective facemask sampling device, in accordance with examples described herein;

FIG. 3 is a flowchart of a method for real-time virus and damaging agent detection, in accordance with examples described herein;

FIG. 4 is a flow chart of a method for training a machine-learning model for virus detection, in accordance with examples described herein;

FIG. 5 is a flow chart of a method for training a first feature detection model and a second feature detection model, in accordance with examples described herein; and

FIG. 6 is a flowchart of a method for real-time virus and damaging agent detection, in accordance with examples described herein.

### DETAILED DESCRIPTION

The present disclosure includes systems and methods for real-time or otherwise fast (e.g., near real-time) virus and damaging agent detection, and more specifically, for capturing images of viruses and other damaging agents in real-time and utilizing such information to detect the presence of viruses and other damaging agents.

For example, a sampling device may generate a digital pattern of a molecular sample (e.g., a blood cell, and air particle, a saliva sample, etc.), where the digital pattern may be the result of the sampling device applying an electron emission to the molecular sample. A computing device having a virus and damaging agent detection machine-learning model and communicatively coupled to the sampling device may receive the digital pattern from the sampling device, analyze the digital pattern, and based on the analysis exceeding an identification threshold, identify a particular damaging agent within the molecular sample. In some examples, the virus and damaging agent detection machine-learning model may be trained using a plurality of output images based on a three-dimensional (3D) model of a particular virus or damaging agent. In some examples, the virus and damaging agent detection machine-learning model may comprise a first feature detection model and a second feature detection model. In some examples, the first feature detection model is trained using at least the plurality of output images to detect a first feature within a digital pattern. In some examples, the second feature detection model is trained using at least the plurality of output images to detect a second feature within a digital pattern.

In some examples, based on the detection of a particular damaging agent, an alert may be sent to a user device that includes information about the particular damaging agent within the molecular sample, as well as time stamp information and geolocation information about the molecular sample. As the spread of damaging agents (e.g., viruses, bacteria, and other contaminants) becomes a cause of global concern, such use of artificial intelligence and machine-learning enables the scientific and medical communities to better identify and track particular damaging agents in a non-invasive, near real-time and accurate manner.

Currently, methods for detecting and/or identifying damaging agents within molecular samples include manually collecting a sample, shipping the sample to an oftentimes off-premise laboratory for various tests, using expensive equipment to manually conduct the various tests on the molecular sample, waiting for the results tests to be shipped back, and attempting to decipher the test results. While time-tested, these traditional methods often suffer from many drawbacks, including that they are often inaccurate, time consuming, expensive, and in some instances, they do not take advantage of advancements in technology. They additionally suffer from the inability to record, track, and plot, current instances of identified damaging agents as well as predict the spread of the identified damaging agents throughout, for example, a community.

Techniques described herein include a damaging agent detection system for real-time virus (or other damaging agent) detection. In some instances, the system may include a sampling device, a computing device comprising a virus and damaging agent detection machine-learning model, and a user device.

The sampling device may generate a digital pattern of a molecular sample and send the digital pattern of the molecular sample to the computing device for damaging agent detection and/or identification. In some examples, the digital pattern may be the result of the sampling device applying an electron emission to the molecular sample. In some examples, the digital pattern may include time stamp information indicative of a time at which the molecular sample was captured by the sampling device and/or geolocation information indicative of a global position or other location information at which the molecular sample was captured by the sampling device.

The computing device may be communicatively coupled to the sampling device and may comprise a virus and damaging agent detection machine-learning model trained to detect and/or identify viruses and/or damaging agents using the digital pattern of the molecular sample. As should be appreciated, and as used herein, AI is meant to encompass machine-learning and other related technologies. In some instances, the computing device may receive the digital pattern of the molecular sample from the sampling device. In some instances, the virus and damaging agent detection machine-learning model of the computing device may analyze the received digital pattern of the molecular sample. Based on the analysis exceeding an identification threshold, the virus and damaging agent detection machine-learning model of the computing device may identify a particular damaging agent within the molecular sample.

The virus and damaging agent detection machine-learning model of the computing device may be trained to detect and/or identify damaging agents within the digital pattern of the molecular sample, using various methods. In some instances, the virus and damaging agent detection machine-learning model may be trained by generating a three-dimensional (3D) model of a particular damaging agent in a particular environment. Using the 3D model, various output images may be captured that include different characteristics, such as different orientations, brightness levels, magnification, or the like, relative to the 3D model. As a specific example, the 3D model may be input into a gaming engine, such as UNITY®, and using the virtual cameras within the gaming environment generated by the gaming engine, the engine may capture output images from different positions, at different brightnesses, etc. of the 3D model.

Additionally, variants of the 3D model of the particular damaging agent may be generated using, for example, image augmentation. In some examples, each variant of the 3D model comprises the 3D model having different shapes, textures, dimensions, stickiness levels, or combinations thereof. Supplemental 3D models for each variant of the 3D model may be generated by executing a script, where each supplemental 3D model comprises a version of each variant of the 3D model at various orientations, rotations, angles, brightness levels, magnification levels, or combinations thereof. In some instances, photorealistic textures may be applied to each supplemental 3D model of each variant of the 3D model of the particular damaging agent. A plurality of output images may be generated, where each supplemental 3D model of each variant of the 3D model corresponds to an output image of the plurality of output images. Using the plurality of output images, the virus and damaging agent detection machine-learning model may be trained to detect the damaging agent from the digital pattern of the molecular sample.

In some examples, the virus and damaging agent detection machine-learning model may comprise a plurality of feature detection models, including in some instances a first feature detection model and a second feature detection model. In some examples, using the plurality output images, the first feature detection model may be trained to detect a first feature of a particular damaging agent within a digital pattern. In some examples, using the plurality output images, the second feature detection model may be trained to detect a second feature of a particular damaging agent within a digital pattern. In some examples, based on the first feature detection model detecting the first feature within the digital pattern and the second feature detection model detecting the second feature within the digital pattern, the virus and damaging agent detection machine-learning model may identify the particular damaging agent within the digital pattern.

In some examples, detecting the first feature within the digital pattern is based at least on the digital pattern exceeding a first identification threshold, where the first identification threshold is associated with the first feature. In some examples, detecting the second feature within the digital pattern is based at least on the digital pattern exceeding a second identification threshold, wherein the second identification threshold is associated with the second feature. As should be appreciated, in some examples, the virus and damaging agent detection machine-learning model may comprise additional, fewer, or different feature detection models than described herein, and description of the first and the second feature detection models is in no way limiting.

In some examples, the computing device may send an alert to a user device communicatively coupled to the computing device, based on identifying the presence of the particular damaging agent, where the alert is indicative of the presence of the identified particular damaging agent. In some instances, the alert may include time stamp information and/or geolocation information. In some examples, based on identifying the presence of the particular damaging agent, the computing device may send an alert to a mapping platform capable of recording and plotting a time and a location from which the molecular sample having the identified particular damaging agent was taken.

In this way, techniques described herein allow for the unobtrusive, accurate, and timely (e.g., near real-time) detection of damaging agents within a molecular sample, as well as the accurate mapping and/or plotting of such identified damaging agents using time stamp and/or geolocation information.

In some embodiments, the systems and methods disclosed herein may receive information related to samples of a damaging agent. The samples may be collected by any of a variant of sampling devices disclosed herein. In some embodiments, a sampling device may be incorporated with a protective facemask in a protective facemask sampling device. One or more processing elements, such as a processing element associated with a server, may receive sample information. The sample information may be indicative of the presence or absence of a damaging agent. The processing element may compare the received sample information to information stored and/or cataloged in a database related to known or likely damaging agents (e.g., a biological or chemical threat database). The threat database may also include information such as location, time, weather, population density, demographic information, etc. that can be used to model, map, or otherwise analyze known, and/or novel damaging agents.

In some embodiments, the systems and methods disclosed herein may automatically generate, via one or more processors, maps models or other representations of a threat from a damaging agent based on the received sample information and/or the threat data store. For example, the systems may generate maps of areas of high transmission of a contagion, or concentration of a chemical threat “i.e., hot zones.” In some embodiments, the systems may generate epidemiological transmission maps, models, or predictions that can forecast the spread and possible impacts of a damaging agent. For example, the systems may generate time-varying and location-specific models showing how a disease may spread, how many people may fall ill, how many people may be hospitalized, and/or how many may die. In some embodiments, the systems and methods may be used to detect, model, map, analyze, and/or control diseases found in either wild or domesticated animal populations such as avian or swine influenza, foot-and-mouth disease, chronic wasting disease, etc.

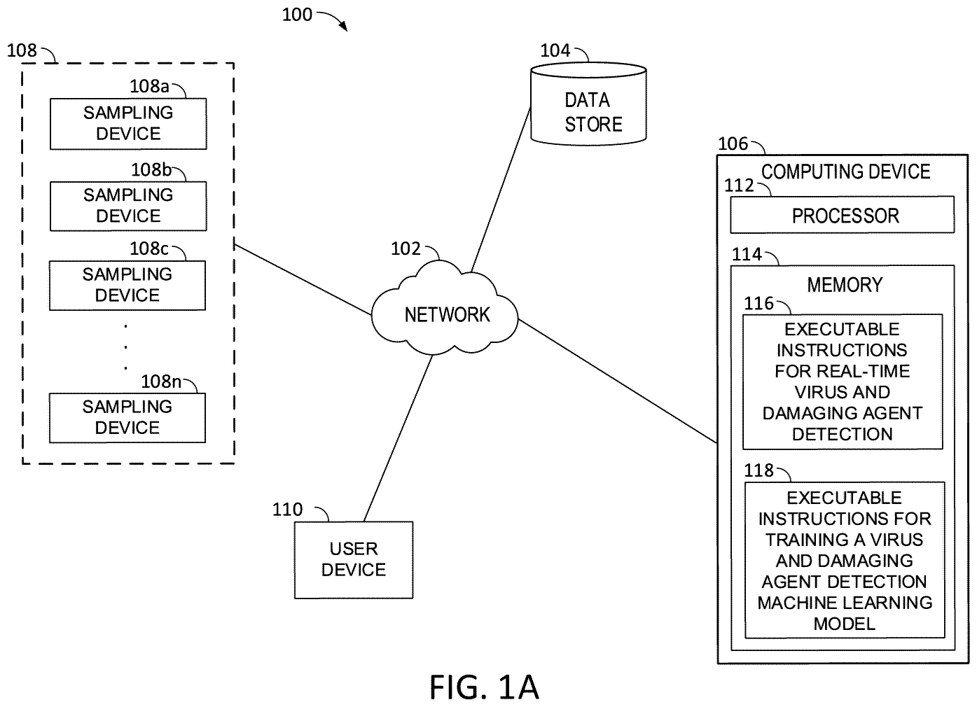

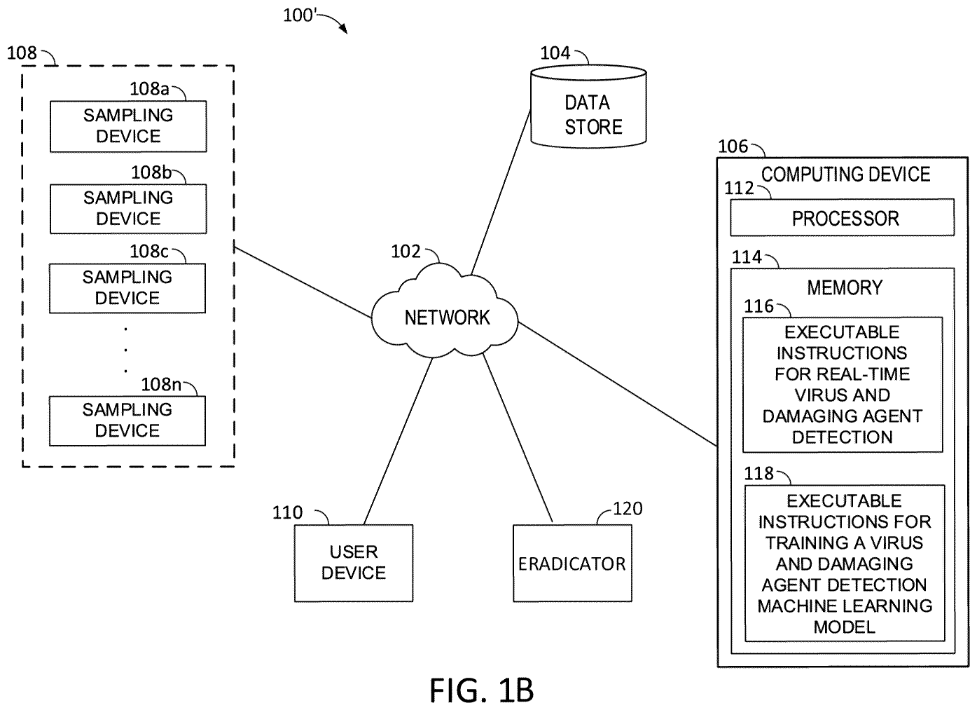

Turing to the figures, FIG. 1A is a schematic illustration of a system 100 for real-time virus and damaging agent detection, in accordance with examples described herein. It should be understood that this and other arrangements and elements (e.g., machines, interfaces, function, orders, and groupings of functions, etc.) can be used in addition to or instead of those shown, and some elements may be omitted altogether. Further, many of the elements described herein are functional entities that may be implemented as discrete or disturbed components or in conjunction with other components, and in any suitable combination and location. Various functions described herein as being performed by one or more components may be carried out by firmware, hardware, and/or software. For instance, and as described herein, various functions may be carried out by a processor executing instructions stored in memory.

System 100 of FIG. 1A includes data store 104 (e.g., a non-transitory storage medium), computing device 106, sampling device 108a, 108b, 108c, and 108n (herein collectively known as sampling devices 108), and user device 110. Computing device 106 includes processor 112, and memory 114. Memory 114 includes executable instructions for real-time virus and damaging agent detection 116, and executable instructions for training a virus and damaging agent detection machine-learning model 118. It should be understood that system 100 shown in FIG. 1A is an example of one suitable architecture for implementing certain aspects of the present disclosure. Additional, fewer, and/or alternative components may be used in other examples.

It should be noted that implementations of the present disclosure are equally applicable to other types of devices such as mobile computing devices and devices accepting gesture, touch, and/or voice input. Any and all such variations, and any combinations thereof, are contemplated to be within the scope of implementations of the present disclosure. Further, although illustrated as separate components of computing device 106, any number of components can be used to perform the functionality described herein. Although illustrated as being a part of computing device 106, the components can be distributed via any number of devices. For example, processor 112 may be provided by one device, server, or cluster of servers, while memory 114 may be provided via another device, server, or cluster of servers.

As shown in FIG. 1A, computing device 106, sampling devices 108, and user device 110 may communicate with each other via network 102, which may include, without limitation, one or more local area networks (LANs), wide area networks (WANs), cellular communications or mobile communications networks, Wi-Fi networks, and/or BLUETOOTH® networks. Such networking environments are commonplace in offices, enterprise-wide computer networks, laboratories, homes, intranets, and the Internet. Accordingly, network 102 is not further described herein. It should be understood that any number of computing devices, sampling devices, and/or user devices may be employed within system 100 within the scope of implementations of the present disclosure. Each may comprise a single device or multiple devices cooperating in a distributed environment. For instance, computing device 106 could be provided by multiple server devices collectively providing the functionality of computing device 106 as described herein. Additionally, other components not shown may also be included within the network environment.

Computing device 106, sampling devices 108, and user device 110 may have access (via network 102) to at least one data store repository, such as data store 104, which stores data and metadata associated with training a virus and damaging agent detection machine-learning model and detecting and or identifying damaging agents within a molecular sample using the virus and damaging agent detection machine-learning model. For example, data store 104 may store data and metadata associated with at least one molecular sample (e.g., a collected molecular sample, a received molecular sample, etc.), time stamp information associated with molecular sample, geolocation information associated with molecular sample, and a digital pattern generated using an electron emission applied to the molecular sample.

Data store 104 may further store data and metadata associated with 3D models of particular damaging agents (or non-damaging agents), variants of 3D models of particular damaging agents, supplemental 3D models comprising versions of each variant of the 3D model of particular damaging agents, as well as orientation data, rotation data, angle data, brightness level data, magnification level data, photorealistic texture data, and the like that may be applied to the 3D models to generate variants of 3D models of particular damaging agents and/or supplemental 3D models comprising version of each variant of the 3D model of particular damaging agents. In some examples, data store 104 may further store data and metadata associated with images of various damaging agents from which 3D models (or 2D models) are generated, such as, for example, images of damaging agents from the U.S. National Institute of Health (NIH). In some examples, data store 104 may store data and metadata associated with images, 3D models, and/or 2D models not related to viruses or damaging agents that may be used to train machine-learning models described herein where identification may be desired.

Data store 104 may further store data and metadata associated with output images associated with 3D models of particular damaging agents, variants of 3D models of particular damaging agents, supplemental 3D models comprising versions of each variant of the 3D model of particular damaging agents, that in some examples, may be used to train the virus and damaging agent detection machine-learning model, and/or a plurality of feature detection models, such as a first feature detection model and a second feature detection model. Data store 104 may further store data and metadata associated with various features associated with 3D models of particular damaging agents, variants of 3D models of particular damaging agents, supplemental 3D models comprising versions of each variant of the 3D model of particular damaging agents, such as, for example, structure (e.g., base structure, membrane structure, etc.), barbs (e.g., surface proteins), and/or other features of a virus or damaging agent.

In implementations of the present disclosure, data store 104 is configured to be searchable for the data and metadata stored in data store 104. It should be understood that the information stored in data store 104 may include any information relevant to real-time virus and damaging agent detection, such as training a virus and damaging agent detection machine-learning model and detecting damaging agents within a molecular sample using the virus and damaging agent detection machine-learning model. For example, data store 104 may include a digital pattern of a molecular sample, including associated time stamp information and geolocation information. In other examples, data store 104 may include 3D model information associated with various damaging agents. In further examples, data store 104 may include image augmentation information for generating variants of the 3D models associated with various damaging agents, such as orientation data, rotation data, angle data, brightness level data, magnification level data, and photorealistic texture data.

Such information stored in data store 104 may be accessible to any component of system 100. The content and the volume of such information are not intended to limit the scope of aspects of the present technology in any way. Further, data store 104 may be a single, independent component (as shown) or a plurality of storage devices, for instance, a database cluster, portions of which may reside in association with computing device 106, sampling devices 108, user device 110, another external computing device (not shown), another external user device (not shown), another sampling device (not shown), and/or any combination thereof. Additionally, data store 104 may include a plurality of unrelated data repositories or sources within the scope of embodiments of the present technology. Data store 104 may be updated at any time, including an increase and/or decrease in the amount and/or types of stored data and metadata.

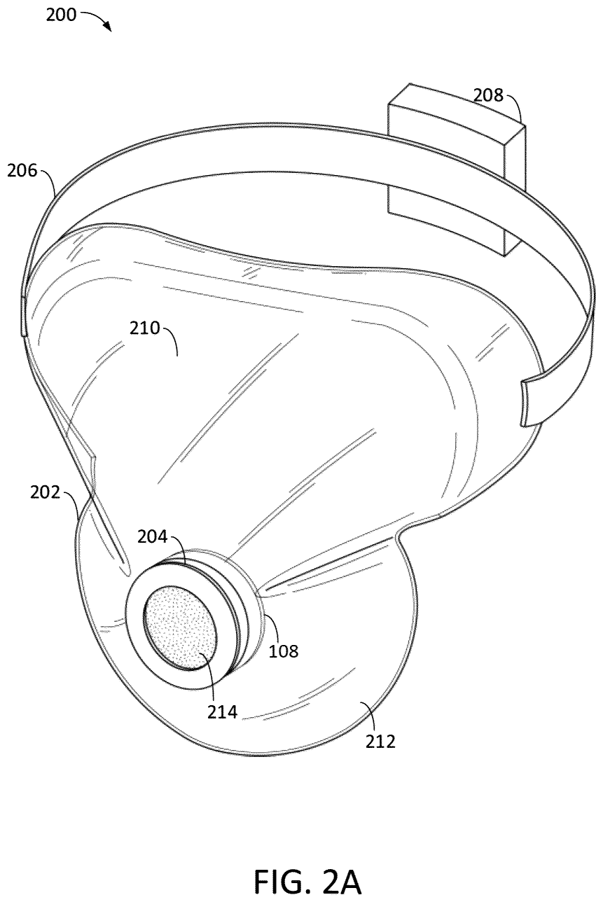

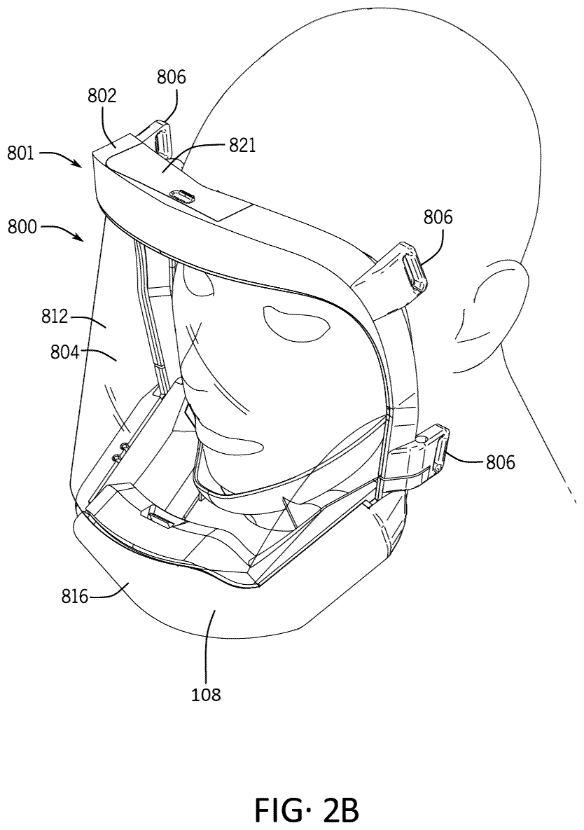

Examples described herein may include sampling devices, such as sampling devices 108. Examples of sampling devices 108 described herein may generally implement the receiving or collecting of a molecular sample, as well as the generation of a digital pattern of the received or collected molecular sample. Examples of sampling devices may include protective face coverings (e.g., protective facemasks, protective face shields), such as sampling device 200 of FIG. 2A or the sampling device 800 of FIG. 2B, body scanners, or any other device, apparatus, or mechanism capable of collecting and/or receiving a molecular sample and generating a digital pattern of the received or collected molecular sample. As used herein, molecular samples may include blood cells, air particles, saliva samples, other organic or inorganic samples, and the like, that may contain known and/or unknown damaging agents.

In some examples, sampling devices 108 may collect a molecular sample, such as a blood sample, air particle sample, saliva sample, other organic or inorganic samples, and the like. In some examples, sampling devices 108 may receive an already collected molecular sample.

In some examples, sampling devices 108 may include a chromatographic immunoassay for the qualitative detection of antigens of a damaging agent (e.g., influenza, SARS-COV-2, measles, etc.). Sampling devices 108 may be connected to the system 100/100′ (e.g., in electronic communication) and/or the system may receive indirect data from a sampling device 108 not associated with the system. Sampling devices 108 may use a chemical reagent that changes color or some other property in the presence of the antigen. The sampling device may reveal a pattern responsive to the reaction of the reagent with the damaging agent (e.g., may form a shape symbol, or colored/tinted area). A sensor, such as an optical sensor, may detect the color change in response to a positive detection of a damaging agent. The sensor may generate a signal suitable to be received by a processor such as a processor 112 discussed herein, indicative of the presence of a damaging agent. In some examples, sampling devices may use a polymerase chain reaction (“PCR”) test to detect genetic material (e.g., RNA or DNA) of the damaging agent.

In some examples, sampling devices 108 may generate a digital pattern of a collected or received molecular sample. In some examples, generating the digital pattern of a molecular sample may be based on the sampling device applying an electron emission to the molecular sample. In some examples, applying the electron emission comprises shooting electrons at a fluorescent screen, where the result of the electron emission creates a negative image on the florescent screen. In some examples, the negative image created as a result of the electron emission is representative of a shape of a particular damaging agent within the molecular sample, where the digital image is indicative of the negative image. As one example, sampling devices 108 may include an electron gun or similar device and/or mechanism configured to apply the electron emission to the molecular sample to generate the digital pattern.

In some examples, sampling devices 108 may include global positioning system (GPS) (or other location) capabilities, time stamp capabilities, or combinations thereof, capable of capturing geolocation (or other location) information, time stamp information, or combinations thereof. In some examples, the digital sample generated by sampling devices 108 may include geolocation information indicative of a global position at which the molecular sample was captured (e.g., collected). In some examples, the digital sample generated by sampling devices 108 may include time stamp information indicative of a time at which the molecular sample was captured (e.g., collected).

As described herein, sampling devices 108 may be communicatively coupled to computing device 106, and may further communicate with other components within system 100 of FIG. 1A using, for example, network 102. As should be appreciated, sampling device 108a, 108b, 108c, and 108n may be communicatively coupled to each other.

While not shown, in some examples, sampling devices 108 may include a sanitizing agent emitter capable of emitting a sanitizing agent configured to neutralize (or destroy) an identified damaging agent.

Examples described herein may include user devices, such as user device 110. User device 110 may be communicatively coupled to various components of system 100 of FIG. 1A, such as, for example, computing device 106 and/or sampling devices 108. User device 110 may include any number of computing devices, including a head mounted display (HMD) or other form of AR/VR headset, a controller, a tablet, a mobile phone, a wireless PDA, touchless-enabled device, other wireless (or wired) communication device, or any other device capable of executing machine-language instructions. Examples of user device 110 described herein may generally implement receiving an alert from computing device 106 indicative of the presence of an identified particular damaging agent within a molecular sample. In some instances, the alert may comprise time stamp information, geolocation information, or combinations thereof.

Examples described herein may include computing devices, such as computing device 106 of FIG. 1A. Computing device 106 may in some examples be integrated with one or more sampling devices, such as sampling devices 108, and/or one or more user devices, such as user device 110, described herein. In some examples, computing device 106 may be implemented using one or more computers, servers, smart phones, smart devices, tables, and the like. Computing device 106 may implement real-time virus and damaging agent detection using a virus and damaging agent detection machine-learning model. As described herein, computing device 106 includes processor 112 and memory 114. Memory 114 includes executable instructions for real-time virus and damaging agent detection 116, and executable instructions for training a virus and damaging agent detection machine-learning model 118, which may be used to implement real-time virus and damaging agent detection. In some embodiments, computing device 106 may be physically coupled to sampling devices 108 and/or user device 110. In other embodiments, computing device 106 may not be physically coupled to sampling devices 108 and/or user device 110 but collocated with the sampling devices and/or the user device. In even further embodiments, computing device 106 may neither be physically coupled to sampling devices 108 and/or user device 110 nor collocated with the sampling devices and/or the user device.

Computing devices, such as computing device 106 described herein may include one or more processors, such as processor 112. Any kind and/or number of processor may be present, including one or more central processing unit(s) (CPUs), graphics processing units (GPUs), other computer processors, mobile processors, digital signal processors (DSPs), microprocessors, computer chips, and/or processing units configured to execute machine-language instructions and process data, such as executable instructions for real-time virus and damaging agent detection 116 and executable instructions for training a virus and damaging agent detection machine-learning model 118.

Computing devices, such as computing device 108, described herein may further include memory 114. Any type or kind of memory may be present (e.g., read only memory (ROM), random access memory (RAM), solid-state drive (SSD), and secure digital card (SD card)). While a single box is depicted as memory 114, any number of memory devices may be present. Memory 114 may be in communication (e.g., electrically connected) with processor 112.

Memory 114 may store executable instructions for execution by the processor 112, such as executable instructions for real-time virus and damaging agent detection 116 and executable instructions for training a virus and damaging agent detection machine-learning model 118. Processor 112, being communicatively coupled to sampling devices 108 and user device 110, and via the execution of executable instructions for real-time virus and damaging agent detection 116 and executable instructions for training a virus and damaging agent detection machine-learning model 118, may detect a damaging agent within a molecular sample using the digital pattern of the molecular sample, and send an alert to, for example, user device 110 indicative of the identification of the particular damaging agent.

In operation, to identify a particular damaging agent within a molecular sample using a digital pattern, processor 112 of computing device 106 may execute executable instructions for real-time virus and damaging agent detection 116 to receive from a sampling device, such as sampling devices 108, the digital pattern of the molecular sample. As described herein, the molecular sample may include a blood sample, an air particle sample, a saliva sample, other organic or inorganic sample, or combinations thereof. In some examples, the molecular sample may include time stamp information and/or geolocation information indicative of the time and/or global position at which the molecular sample was taken.

Processor 112 of computing device 106 may execute executable instructions for real-time virus and damaging agent detection 116 to analyze the received digital pattern.

Processor 112 of computing device 106, may execute executable instructions for real-time virus and damaging agent detection 116 to, based on the analyzing exceeding an identification threshold, identify a particular damaging agent within the molecular sample. In some examples, the particular damaging agent may be a virus, bacterium, parasite, protozoa, prion, or combinations thereof. In some examples, the particular damaging agent may be a severe acute respiratory syndrome coronavirus 2 (SARS-COV-2) virus that causes coronavirus disease 19 (COVID-19). In some examples, the particular damaging agent may be unknown, unrecognizable, or otherwise unidentifiable.

In some examples, processor 112 of computing device 106 may execute executable instructions for real-time virus and damaging agent detection 116 to send an alert to a user device, such as user device 110, based on identifying and/or detecting the presence of the particular damaging agent within the molecular sample. In some examples, the alert may include time stamp information, geolocation information, or combinations thereof.

In some examples, processor 112 of computing device 106 may execute executable instructions for real-time virus and damaging agent detection 116 to, based on identifying and/or detecting the presence of the particular damaging agent, may send an alert including time stamp information and/or geolocation information to a mapping platform (or social media platform) capable of recording and/or plotting a time and a location from which the molecular sample having the identified particular damaging agent was taken. In some examples, the mapping platform, including the time and location data, may be used to predict the spread of the identified particular damaging agent. In some examples, alerts relating to a predicted spread of the identified particular damaging agent may be sent to a user device, or a sampling device.

As should be appreciated, while 3D models are discussed herein with respect to detecting and/or identify particular damaging agents within a molecular sample via the analysis and exceeding an identification threshold, additional and/or alternative models, such as two dimensional (2D) models may also be used to detect and/or identify particular damaging agents within a molecular sample. Further, the discussion herein of using 3D models for use detection and/or identification is in no way meant to be limiting, and use of 2D models is contemplated to be within the scope of this disclosure.

As discussed herein, to identify and/or detect a particular damaging agent from a digital pattern of a molecular sample, computing device may use a virus and damaging agent detection machine-learning model. In operation, to train a virus and damaging agent detection machine-learning model of a computing device, such as computing device 106, processor 112 of computing device 106 may, by executing executable instructions for training a virus and damaging agent detection machine-learning model 118, generate a three dimensional (3D) model of a particular damaging agent in a particular environment. By way of example, processor 112 of computing device 106 may generate a 3D model of streptococcal pharyngitis (the bacteria that causes strep throat) in saliva. In another example, processor 112 of computing device 106 may generate a 3D model of severe acute respiratory syndrome coronavirus 2 (the virus that causes COVID-19) in a blood sample. In yet another example, processor 112 of computing device 106 may generate a 3D model of Plasmodium (the protozoan that causes malaria) in an air particle.

In some examples, a 3D model may be generated using a high-powered computer-graphics engine, such as a video-gaming engine capable of generating and/or rendering 3D models with low latency and high resolution. In some examples, a 3D model may be generated using other computer-graphics engines.

In some examples, the 3D model may be generated using images of damaging agents, such as images from the U.S. National Institute of Health (NIH), which may be stored, for example, in a data store, such as data store 104.

Processor 112 of computing device 106 may, by executing executable instructions for training a virus and damaging agent detection machine-learning model 118, generate variants of the 3D model of the particular damaging agents using image augmentation. In some examples, and using image augmentation, each variant of the 3D model of the particular damaging agent may have a different shape (e.g., barrier, structure), stickiness level, or combinations thereof, from each other. Continuing with an example discussed above, processor 112 of computing device 106 may, by executing executable instructions for training a virus and damaging agent detection machine-learning model 118, generate variants of the 3D model of SARS-COV-2. In some examples, a variant 3D model of SARS-COV-2 may be stickier than the original 3D model. In some examples, a variant 3D model of SARS-COV-2 may be less sticky than the original 3D model. In some examples, a variant 3D model of SARS-COV-2 may be a different, more oval shape than the original 3D model. In some example, a variant 3D model of SARS-COV-2 may be an asymmetrical shape.

Processor 112 of computing device 106 may, by executing executable instructions for training a virus and damaging agent detection machine-learning model 118, execute a script that generates a plurality of supplemental 3D models for each variant of the 3D model of the particular damaging agent. In some examples, each supplement 3D model may comprise a different version of the variant of the 3D model at various orientations, rotations, angles, brightness levels, magnification levels, or combinations thereof. Continuing with the same example as above, in one example, a supplemental 3D model may include an asymmetrically shaped 3D model of SARS-COV-2 with a magnification level of 80%. In another example, a supplemental 3D model may include an asymmetrically shaped 3D model of SARS-COV-2 with a brightness level of 35%. In another example, a supplemental 3D model may include an asymmetrically shaped 3D model of SARS-COV-2 rotated 65 degrees.

It should be noted that in some instances, the processor 112 may not generate separate 3D models as variants, but rather may capture output images of a single 3D model, but with different camera characteristics, e.g., at different angles relative to the 3D model or 3D object, different orientations, different magnification levels, different brightness, and so on.

Processor 112 of computing device 106 may, by executing executable instructions for training a virus and damaging agent detection machine-learning model 118, apply photorealistic textures to each (or to some, or to one) supplemental 3D model of each variant of the 3D model of the particular damaging agent. As one example, processor 112 of computing device 106 may apply a photorealistic texture to the asymmetrically shaped 3D model of SARS-COV-2 with a magnification level of 80%. In another example, processor 112 of computing device 106 may apply a photorealistic texture to the supplemental 3D model may include an asymmetrically shaped 3D model of SARS-COV-2 with a brightness level of 35%. In another example, processor 112 of computing device 106 may apply a photorealistic texture to the asymmetrically shaped 3D model of SARS-COV-2 rotated 65 degrees.

In some examples, the photorealistic textures applied to the supplemental 3D models of the variants of the 3D model of the particular damaging agent may be varied. For example, the photorealistic textures may correspond to various conditions of a virus or a damaging agent. In some examples, the photorealistic textures may correspond to various states of the virus or the damaging agent. In some examples, the photorealistic textures may be relevant to the virus or the damaging agent to which it is being applied. In some examples, the photorealistic textures may be relevant to the objects other than a virus or a damaging agent. In some examples, the photorealistic textures applied may be extracted from one or more images showing photorealistic textures.

Processor 112 of computing device 106 may, by executing executable instructions for training a virus and damaging agent detection machine-learning model 118 and based on each supplemental 3D model of each variant of the 3D model of the particular damaging agent, generate a plurality of output images, where each supplemental 3D model of each variant of the 3D model corresponds to an output image of the plurality of output images.

Processor 112 of computing device 106 may, by executing executable instructions for training a virus and damaging agent detection machine-learning model 118 and using the plurality of output images, train the virus and damaging agent detection machine-learning model to detect the damaging agent from digital pattern of the molecular sample.

As should be appreciated, while processor 112 may use variants of a 3D model and supplemental 3D models of variants of a 3D model to train the virus and damaging agent detection machine-learning model as discussed herein, in some examples, processor 112 may train the virus and damaging agent detection machine-learning model using a single 3D model of a particular damaging agent in a particular environment. In some examples, utilizing the 3D mode, a plurality of output images may be generated, where the plurality of output images are captured at one or more of different rotations of the 3D model, varying brightness levels of the 3D model, varying magnification levels of the 3D model, or combinations thereof. Using the plurality of output images, the virus and damaging agent detection machine-learning model may be trained to detect a damaging agent from a digital pattern of a molecular sample.

As should be appreciated, while 3D models are discussed herein with respect to training the virus and damaging agent detection machine-learning model to detect and/or identify particular damaging agents within a molecular sample, additional and/or alternative models, such as two dimensional (2D) models may also be used to train the virus and damaging agent detection machine-learning model. Further, the discussion herein of using 3D models for use in training the virus and damaging agent detection machine-learning model is in no way meant to be limiting, and use of 2D models is contemplated to be within the scope of this disclosure.

As should further be appreciated, while 3D and 2D models of viruses and damaging agents are discussed herein with respect to training a machine-learning model to detect and/or identify particular viruses and/or other damaging agents within a molecular sample, additional, fewer, and/or alternative 3D and 2D models may be used to train the machine-learning model. For example, 3D and 2D models other than those relating to viruses or other damaging agents may be used to train the machine-learning model. In some instances, 3D and 2D models may be used to train the machine-learning model where identification of objects not related to viruses or other damaging agents is desired. Accordingly, while detection and identification of viruses or other damaging agents is discussed herein, the machine-learning model may be trained to detect and/or identify other features and/or object not related to viruses or other damaging agents, and discussion of viruses or other damaging agents is in no way limiting.

FIG. 1B illustrates an example of a system 100′ similar in many aspects to the system 100. The system 100′ may include an eradicator 120 that can be selectively deployed to eliminate or neutralize a damaging agent. For example, the eradicator 120 may include an autonomous vehicle or aircraft that includes a sanitizing emitter (e.g., an ultraviolet light source) that can emit a sanitizing agent to eliminate or neutralize a damaging agent. In some embodiments, an eradicator may be an airborne of land-based autonomous drone, remote controller vehicle, or the like. One or more eradicators may be deployed to a location where a presence of a damaging agent has been detected as discussed herein.

Turing now to FIGS. 2A and 2B, FIG. 2A illustrates an example protective facemask sampling device 200, in accordance with examples described herein. FIG. 2B illustrates another example of a protective facemask sampling device 800 as discussed herein. Protective facemask sampling devices, such as protective facemask sampling device 200 and/or 800 may generally implement the receiving or collecting of a molecular sample, as well as the generation of a digital pattern of the received or collected molecular sample. Additionally or alternately, the devices 200 and/or 800 may sample the air (inside, outside, or passing through the devices 200/800) for the presence of a damaging agent and generate a signal indicative of the presence of the damaging agent. The protective facemask sampling device 200 includes a protective (e.g., impermeable) membrane 202 having eye cover portion 210 and nose and mouth covering portion 212, respiration aperture chamber 204, attachment membrane 206, and power source 208. Respiration aperture chamber 204 may include electron emitter 214. The protective facemask sampling device 200 may include any sampling device 108 disclosed herein.

With specific reference to FIG. 2B, another example of a mask 800 is shown. The mask 800 may be substantially similar to the mask 200 and/or other mask examples described herein. The mask 800 may include a frame 801 that surrounds the face shield or lens. A seal may be coupled to the frame 801 and extend around a perimeter thereof. The filter cartridge may be coupled to the frame 801 as well. The mask 800 may have a transparent area sufficiently large to allow visibility of the user's face to others with whom the user interacts (e.g., patients). The transparent area may be sufficiently large so as to not obstruct the user's peripheral vision. The protective facemask sampling device may include an electron emitter 214, similar to the mask 200. The protective facemask sampling device 800 may include any sampling device 108 disclosed herein.

The mask 800 may include an impermeable membrane 812 substantially similar to the impermeable membrane 202. In some instances, the impermeable membrane 812 may be fully transparent or at least partially transparent. In some examples, the impermeable membrane 812 or face shield may function as a lens to other viewable element to allow the user's facial features and expressions to be visible to others. The impermeable membrane 812 may be configured to extend away from the user's face. Such an arrangement may increase user comfort and may help reduce fogging of the impermeable membrane 812, such as due to the user's breath, perspiration, or the like. In some embodiments, the lens 812 may be configured to define an extra pocket or space adjacent to a user's mouth and nose, allowing a more comfortable fit and helping to reduce fogging.

The frame 801 may receive other components of the mask 800 such as the seal, a filter cartridge 816, a sanitizing agent source 821 (such as batteries), a cartridge receptacle 820, a sampling device 108, and/or one or more straps (e.g., received in the securing supports 806). The frame 801 and the membrane 812 may form a respiration chamber 804 similar to the respiration chamber 204.

In one example, a user of protective facemask sampling device 200/800 may wear protective facemask sampling device 200/800 and subsequently breathe through the respiration aperture chamber 204/804. Here, respiration aperture chamber 204/804 may collect a molecular sample, such as an air particle sample, from the user breathing. After collecting the air particle sample, electron emitter 214 may apply an electron emission to the air particle sample, resulting in a digital pattern of the air particle sample. Alternately or additionally, a sampling device 108 may determine the presence of a damaging agent, such as via chromatographic immunoassay or PCR. Protective facemask sampling device 200/800 may send the digital pattern of the air particle sample and/or indication of the presence of a damaging agent, along with any associated time stamp information or geolocation information, to a computing device having a virus and damaging agent detection machine learning-model, such as computing device 106 of FIG. 1A-1B, to detect and/or identify the presence of a damaging agent within the air particle sample.

While discussed but not shown, protective facemask sampling device 200 and/or 800 may further include capabilities to communicate with various components of system 100 of FIG. 1A-1B, via network 102. While discussed but not shown, protective facemask sampling device 200/800 may further include global positioning system (GPS) capabilities, time stamp capabilities, or combinations thereof, capable of capturing geolocation information, time stamp information, or combinations thereof of a molecular sample. While discussed but not shown, protective facemask sampling device 200/800 may further include a sanitizing agent emitter capable of emitting a sanitizing agent configured to neutralize (or destroy) an identified damaging agent. In some examples, sampling devices, such as sampling devices 108 of FIG. 1A-1B and protective facemask sampling device 200/800 of FIG. 2A/B may be configured to receive alerts from a computing device, such as computing device 106 of FIG. 1A-1B indicative of the presence of a particular damaging agent.

As should be understood, while protective facemask sampling devices 200/800 includes various features, other protective facemask sampling devices may include additional, alternative, and/or fewer features, and that the features discussed with respect to protective facemask sampling device 200/800 are in no way limiting.

As should be further understood, and as described herein, while FIG. 2A/B illustrate example protective facemask sampling devices 200/800, other types of sampling devices are contemplated to be within the scope of this disclosure, and discussion of protective facemask sampling device 200/800 is in no limiting. For example, and as discussed herein, additional and/or alternative types of sampling devices may include protective face shields, body scanners, or any other device, apparatus, or mechanism (wearable or non-wearable) capable of collecting and/or receiving a molecular sample and generating a digital pattern of the received or collected molecular sample. Such additional and/or alternative sampling devices may include, for example, global positioning system (GPS) capabilities, time stamp capabilities, or combinations thereof, capable of capturing geolocation information, time stamp information, or combinations thereof of a molecular sample. Such additional and/or alternative sampling devices may further include, for example, sanitizing agent emitter(s) capable of emitting a sanitizing agent configured to neutralize (e.g., deactivate, sanitize, eradicate, destroy, etc.) an identified damaging agent.

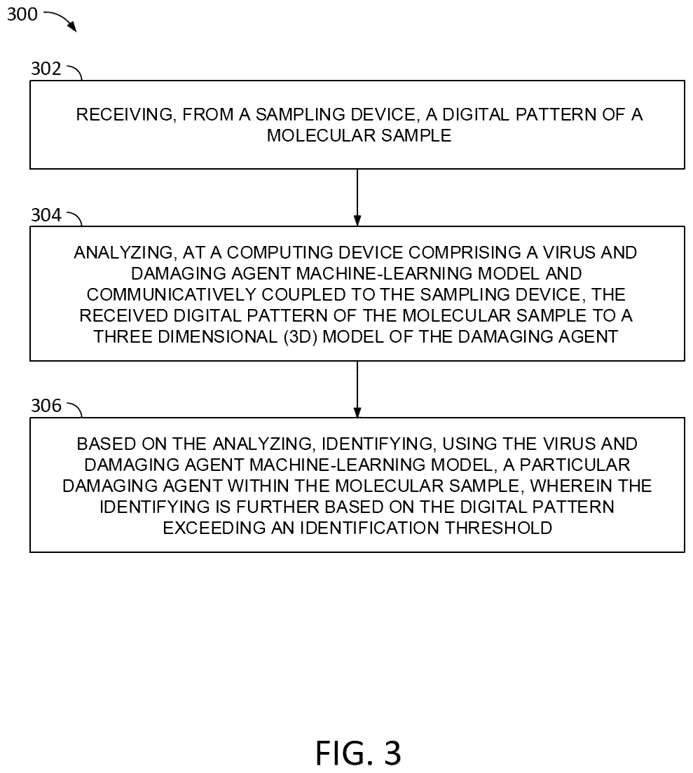

Now turning to FIG. 3, FIG. 3 is a flowchart of a method 300 for real-time virus and damaging agent detection, in accordance with examples described herein. The method 300 may be implemented, for example, using the system 100 of FIG. 1A-1B.

The method 300 includes receiving, from a sampling device, a digital pattern of a molecular sample in step 302; analyzing, at a computing device comprising a virus and damaging agent machine-learning model and communicatively coupled to the sampling device, the received digital pattern of the molecular sample to a three dimensional (3D) model of the damaging agent in step 304; and based on the analyzing, identifying, using the virus and damaging agent machine-learning model, a particular damaging agent within the molecular sample, wherein the identifying is further based on the digital pattern exceeding an identification threshold in step 306.

Step 302 includes receiving, from a sampling device, a digital pattern of a molecular sample. As described herein, the molecular sample may include a blood sample, an air particle sample, a saliva sample, other organic or inorganic sample, or combinations thereof. In some examples, the molecular sample may include time stamp information and/or geolocation information indicative of the time and/or global position at which the molecular sample was taken.

Step 304 includes analyzing, at a computing device comprising a virus and damaging agent machine-learning model and communicatively coupled to the sampling device, the received digital pattern of the molecular sample to a three dimensional (3D) model of the damaging agent.

Step 306 based on the analyzing, identifying, using the virus and damaging agent machine-learning model, a particular damaging agent within the molecular sample, wherein the identifying is further based on the digital pattern exceeding an identification threshold. In some examples, the particular damaging agent may be a virus, bacterium, parasite, protozoa, prion, or combinations thereof. In some examples, the particular damaging agent may be a severe acute respiratory syndrome coronavirus 2 (SARS-COV-2) virus that causes coronavirus disease 19 (COVID-19).

In some examples, the particular damaging agent may be unknown, unrecognizable, or otherwise unidentifiable (e.g., novel, new, etc.). In some examples, and as discussed herein, a first feature detection model may be trained to identify and/or detect a first feature of a digital pattern, and a second feature detection model may be trained to identify and/or detect a second feature of a digital pattern. In some examples, the first feature detection model may detect the first feature, but the second feature detection model may not detect the second feature. In some examples, based on the first feature detection model detecting the first feature, and the second feature detection model not detecting the second feature, the virus and damaging agent detection machine-learning model may determine the digital pattern is of an unknown virus, damaging agent, or other object. By utilizing the different feature detection models, however, the system may determine that the damaging agent is related (e.g., a type of corona virus), to other known damaging agents. In some embodiments, when a novel or unknown damaging agent is detected, the system may generate a Day 0 alert that informs authorities of the possibility of a new damaging agent, such that the authorities can take appropriate action to contain or limit the spread thereof. For example, the system may generate an automatic notification to select devices, such as those linked with authorities, or the like.

Similarly, in some examples, the first feature detection model may not detect the first feature, but the second feature detection model may detect the second feature. In some examples, based on the first feature detection model not detecting the first feature, and the second feature detection model detecting the second feature, the virus and damaging agent detection machine-learning model may determine the digital pattern is of an unknown virus, damaging agent, or other object. Further, in some examples, first feature detection model may not detect the first feature, and the second feature detection model may not detect the second feature. In some examples, based on neither the first feature detection model detecting the first feature nor the second feature detection model detecting the second feature, the virus and damaging agent detection machine-learning model may determine the digital pattern is of an unknown virus, damaging agent, or other object.

In some examples, based at least on which feature (e.g., a first feature, a second feature, a third feature, etc.) is detected, the virus and damaging agent machine-learning model may be able to determine a type or category of virus, damaging agent, or object of the digital pattern, rather than the particular virus, damaging agent, or object. As should be appreciated, while the first feature detection model and the second feature detection model are discussed in relation to determining an unknown virus, damaging agent, or other object, such discussion is in no way limiting, and such determinations may be made using additional, fewer, and/or alternative feature detection models, as well as a single virus and damaging agent machine-learning model.

In some examples, the virus and damaging agent machine-learning model may compare (or analyze, evaluate, etc.) the digital pattern to a library, corpus, dataset, and the like comprising 3D and 2D models of viruses, damaging agents, and other objects. In some examples, based at least on the virus and damaging agent machine-learning model determining the digital pattern does not match a 3D or 2D model in the library, the virus and damaging agent machine-learning model may determine the digital pattern is a new virus, new damaging agent, or new other object. As should be appreciated, while damaging agents are discussed herein, that is in no way limiting, and other non-damaging agents are contemplated to be within the scope of this disclosure.

In some examples based on identifying and/or detecting the presence of the particular damaging agent, an alert including time stamp information and/or geolocation information may be sent to a user device. In some examples based on identifying and/or detecting the presence of the particular damaging agent, an alert including time stamp information and/or geolocation information may be sent to a mapping platform (or social media platform) capable of recording and/or plotting a time and a location from which the molecular sample having the identified particular damaging agent was taken.

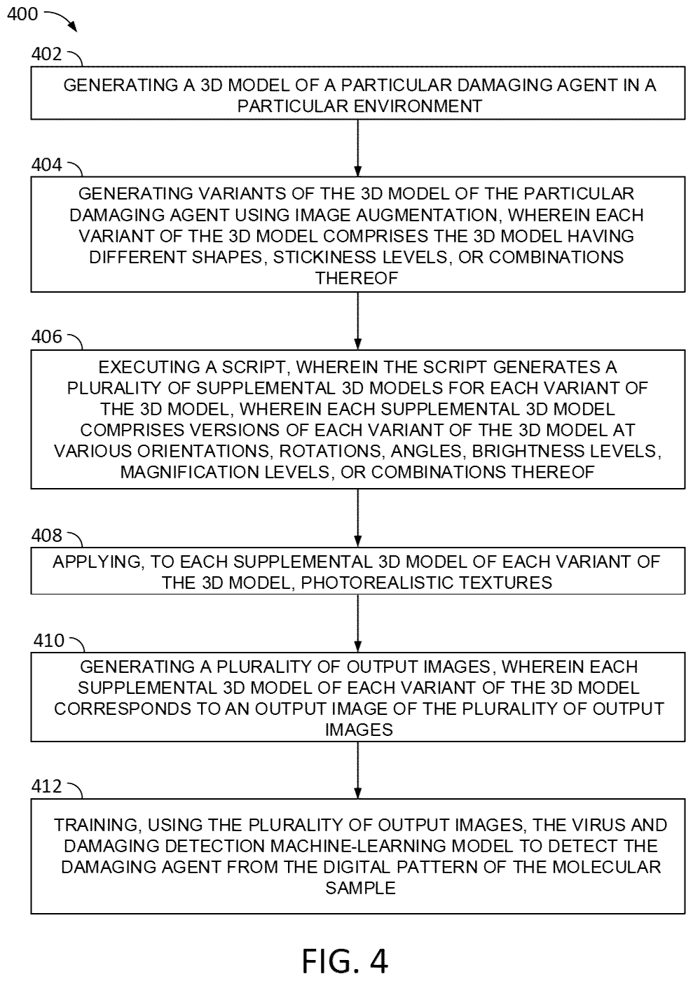

Now turning to FIG. 4, FIG. 4 is a flowchart of a method 400 for training a machine-learning model for virus detection, in accordance with examples described herein. The method 400 may be implemented, for example, using the system 100 of FIG. 1A-1B.

The method 400 includes generating a three dimensional (3D) model of a particular damaging agent in a particular environment in step 402; generating variants of the 3D model of the particular damaging agent using image augmentation, wherein each variant of the 3D model comprises the 3D model having different shapes, stickiness levels, or combinations thereof in step 404; executing a script, wherein the script generates a plurality of supplemental 3D models for each variant of the 3D model, wherein each supplemental 3D model comprises versions of each variant of the 3D model at various orientations, rotations, angles, brightness levels, magnification levels, or combinations thereof in step 406; applying, to each supplemental 3D model of each variant of the 3D model, photorealistic textures in step 408; generating a plurality of output images, wherein each supplemental 3D model of each variant of the 3D model corresponds to an output image of the plurality of output images at step 410; and training, using the plurality of output images, the virus and damaging detection machine-learning model to detect the damaging agent from the digital pattern of the molecular sample in step 412.

Step 402 includes generating a 3D model of a particular damaging agent in a particular environment. In some examples, the 3D model may be generated using a high-powered computer-graphics engine, such as a video-gaming engine capable of generating and/or rendering 3D models with low latency and high resolution. In some examples, a 3D model may be generated using other computer-graphics engines. In some examples, the 3D model may be generated using images of damaging agents, such as images from the U.S. National Institute of Health (NIH), which may be stored, for example, in a data store, such as data store 104.

Step 404 includes generating variants of the 3D model of the particular damaging agent using image augmentation, wherein each variant of the 3D model comprises the 3D model having different shapes, stickiness levels, or combinations thereof. In some examples, and using image augmentation, each variant of the 3D model of the particular damaging agent may have a different shape (e.g., barrier, structure), stickiness level, or combinations thereof, from each other.

Step 406 includes executing a script, wherein the script generates a plurality of supplemental 3D models for each variant of the 3D model, wherein each supplemental 3D model comprises versions of each variant of the 3D model at various orientations, rotations, angles, brightness levels, magnification levels, or combinations thereof. In some examples, each supplement 3D model may comprise a different version of the variant of the 3D model at various orientations, rotations, angles, brightness levels, magnification levels, or combinations thereof.

Step 408 includes applying, to each supplemental 3D model of each variant of the 3D model, photorealistic textures.

Step 410 includes generating a plurality of output images, wherein each supplemental 3D model of each variant of the 3D model corresponds to an output image of the plurality of output images.

Step 412 includes training, using the plurality of output images, the virus and damaging detection machine-learning model to detect the damaging agent from the digital pattern of the molecular sample. As described herein, in some examples, the virus and damaging detection machine-learning model may comprise a plurality of feature detection models, such as a first feature detection model and a second feature detection model. In some examples, the plurality of output images may be used to train the first feature detection model to detect a first feature within a digital pattern. In some examples, the plurality of output images may be used to train the second feature detection model to detect a second feature within a digital pattern.

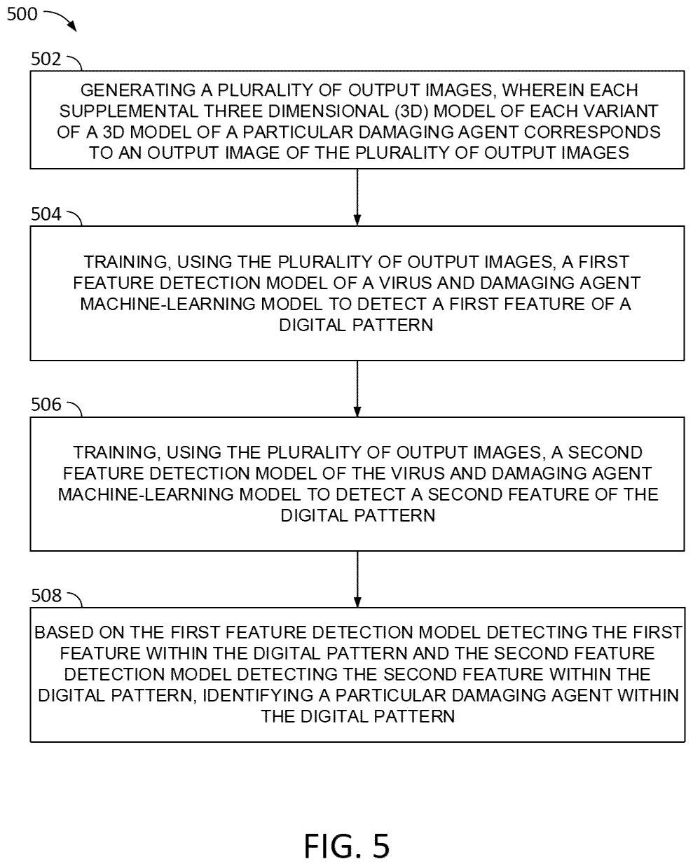

Now turning to FIG. 5, FIG. 5 is a flowchart of a method 500 for training a first feature detection model and a second feature detection model, in accordance with examples described herein. The method 500 may be implemented, for example, using the system 100 of FIG. 1A-1B.

The method 500 includes generating a plurality of output images, wherein each supplemental three dimensional (3D) model of each variant of a 3D model of a particular damaging agent corresponds to an output image of the plurality of output images in step 502; training, using the plurality of output images, a first feature detection model of a virus and damaging agent machine-learning model to detect a first feature of a digital pattern in step 504; training, using the plurality of output images, a second feature detection model of the virus and damaging agent machine-learning model to detect a second feature of the digital pattern in step 506; and based on the first feature detection model detecting the first feature within the digital pattern and the second feature detection model detecting the second feature within the digital pattern, identifying a particular damaging agent within the digital pattern in step 508.

Step 502 includes generating a plurality of output images, wherein each supplemental three dimensional (3D) model of each variant of a 3D model of a particular damaging agent corresponds to an output image of the plurality of output images.

Step 504 includes training, using the plurality of output images, a first feature detection model of a virus and damaging agent machine-learning model to detect a first feature of a digital pattern. In some examples, and as described herein, a first feature may include the structure (e.g., base structure, membrane structure, etc.), the barbs (e.g., surface proteins), and/or other features, of a virus or damaging agent.

Step 506 includes training, using the plurality of output images, a second feature detection model of the virus and damaging agent machine-learning model to detect a second feature of the digital pattern. In some examples, and as described herein, a second feature may include the structure (e.g., base structure, membrane structure, etc.), the barbs (e.g., surface proteins), and/or other features, of a virus or damaging agent.

Step 508 includes, based on the first feature detection model detecting the first feature within the digital pattern and the second feature detection model detecting the second feature within the digital pattern, identifying a particular damaging agent within the digital pattern. In some examples, detecting the first feature within the digital pattern is based at least on the digital pattern exceeding a first identification threshold, wherein the first identification threshold is associated with the first feature. In some examples, detecting the second feature within the digital pattern is based at least on the digital pattern exceeding a second identification threshold, wherein the second identification threshold is associated with the second feature.

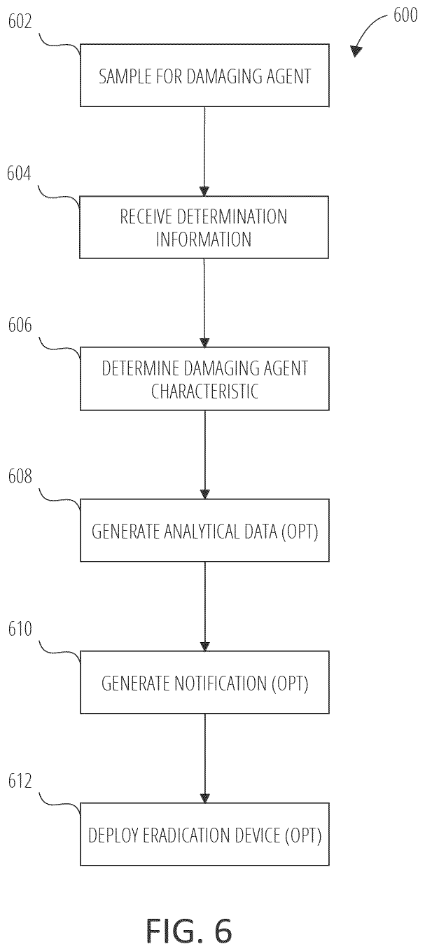

Now turning to FIG. 6, FIG. 6 is a flowchart of a method 600 for real-time virus and damaging agent detection, in accordance with examples described herein. The method 600 may be implemented, for example, using the system 100 of FIG. 1A and/or the system 100′ of FIG. 1B. The steps of the method 600 may be executed in an order other than a shown, and/or one or more steps may be optional. Additionally or alternately, two or more steps may be executed in parallel or on different processing elements.

The method 600 may begin in step 602 and the system 100/100′ receives sample data related to a damaging agent. The sample data may be received by a sampling device 108 samples for the presence of a damaging agent. In some embodiments, the sample data may be received by the system 100/100′ from a sampling device not associated with the system (e.g., a separate or stand-alone sampling device). In some embodiments, the sample may be collected from the air. In other embodiments, the sample may be collected from a surface, soil, biological fluid or tissue, water, etc. The sampling device 108 may be included in a protective facemask sampling device 200/800, or may be a separate device. The sampling device may sample air inside, outside, or passing through a protective facemask sampling device (e.g. a mask 200/800). The sampling device may collect particles of the damaging agent exhaled in a user's breath, captured in a filter media from air inhaled by the user, or ambient air proximate to the protective facemask sampling device 200/800. The sampling device 108 may be triggered based on a breath of the user or may be automatically driven by a processor 112 (e.g., on a timer or other event such as movement).

The method 600 may proceed to step 604 and the system 100/100′ determines the presence of a damaging agent. For example, as disclosed herein, the sampling device 108 may determine the presence of a damaging agent, such as via chromatographic immunoassay or PCR. The sampling device 108 may include a sensor that detects a color change of a portion of the sampling device 108 responsive to a chemical reaction of the damaging agent with a portion of the sampling device 108. The sensor may generate a signal in response to the detection of a damaging agent. For example, the sampling device 108 may form a pattern, symbol, or colored/tinted area responsive to a reagent reacting with the damaging agent. The sensor may be an optical sensor that detects the pattern formed by the sampling device where the pattern is indicative of the presence of the damaging agent. The signal generated by the sensor and indicative of the determination of the presence of the damaging agent may be received by one or more processors, such as the processor 112. The signal may be received by the processor via any wired or wireless communication suitable to transmit such a signal. For example, the processor 112 may be electrically connected to the sensor in the protective facemask sampling device 200/800. In another example, the sensor may be in wireless communication with the processor 112 via a wireless connection, either directly or via a user device 110, a network 102, a wireless network such as a cellular telephone network, a private network, virtual private network, the internet, or the like. Alternately or additionally, the system 100/100′ may determine the presence of the damaging agent based on sample data and/or signal data received by a stand-alone sampling device not associated with the system 100/100′. For example, the system 100/100′ may receive data from a public or private database that includes epidemiological or other data associated with a damaging agent.

The method 600 may proceed to step 606 and the system 100 and/or 100′ determines a characteristic of the damaging agent. For example, the system 100/100′ may determine a type of the damaging agent. For example the system 100/100′ may determine whether the damaging agent is a chemical agent, virus, bacterium, or the like. In another example, the system 100/100′ may determine a sub-type of the damaging agent, such as a type of virus (e.g., SARA-COV-2, or variant thereof, influenza, etc.) For example, the AI or machine learning algorithm may compare data related to the damaging agent, e.g., such as determined by the sampling device 108, to a pattern of characteristics of damaging agents stored in a data store 104, such as a threat data store. The AI or machine learning algorithm may classify the detected damaging agent into one or more categories. If the AI or machine algorithm cannot determine a type of sub-type of the damaging agent, the system 100/100′ may determine that the damaging agent is novel. Such detection of a novel damaging agent, also known as a Day 0 detection, may have the benefit of providing early detection of novel threats such that authorities, such as public health departments, can take appropriate actions to contain the damaging agent. Similarly, the system 100/100′ may determine a mutation to a known damaging agent (e.g., delta, omicron, BA5 or other sub-variants of the SARS-COV-2 virus).

The method may optionally proceed to step 608 and the system 100/100′ automatically generates analytical data related to the damaging agent. In some embodiments, the analytical data may include a map, model, or other related to the damaging agent. For example, the system 100/100′ may use an artificial intelligence or machine learning algorithm and/or other computing modules, such as an analytic algorithms to perform all or a portion of step 608. For example, in some embodiments, the systems 100/100′ may automatically generate, via one or more processors, maps, models or other representations of a threat from a damaging agent based on the received sample information and/or the threat data store. For example, the system 100/100′ may generate a hot zone map, exclusion zone map, safe zone map, predictions, and/or the like based on growth, spread, and origination of the damaging agent. In some embodiments, the system 100/100′ may generate epidemiological, mortality, morbidity, and/or transmission maps, models, or predictions that can forecast the spread and/or possible impacts of a damaging agent. For example, the system 100/100′ may generate time-varying and/or location-specific models showing how a disease may spread, how many people may fall ill, how many people may be hospitalized, and/or how many may die. For example, the system 100/100′ may generate a chart showing a prediction of when spread, illness, infection, and/or deaths from a damaging agent may vary over time at a location, in a city, municipality, state, province, county, country, etc. In some embodiments, the system 100/100′ may generate data to perform contact tracing.

The method 600 may optionally proceed to step 610 and the system 100/100′ generates a notification related to the detected damaging agent. For example, if the system 100/100′ detects the presence of SARS-COV-2, the system 100/100′ may generate a notification on a user device 110 or other device. For example, a notification may include an indication of the type and/or subtype of damaging agent detected, or in the case of a novel damaging agent, may generate a Day 0 alert. The notification may be transmitted to one or more devices, such as personal user devices, devices associated with a health or other authority, or the like. The notification may be displayed locally on a user device, and/or maybe transmitted to another device such as a server, directly or via a wired or wireless network. In some embodiments, the system may automatically perform contact tracing, notifying individuals or organizations of their likely contact with a damaging agent.

The method 600 may optionally proceed to operation 612 and the system 100/100′ may deploy one or more eradicators 120 configured to emit a sanitizing agent configured to deactivate, sanitize, or reduce the danger associated with the damaging agent. For example, the system 100/100′ may deploy one or more eradicators 120 that move to a location where the damaging agent was detected (e.g., as in step 604) and emit ultraviolet light that can deactivate biological threats such as the SARS-COV-2 virus. Step 612 may also include a number of actions taken automatically by the system 100/100′. For example, the system 100/100′ may deploy first responders, vaccines, containment measures, and/or quarantines.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications may be made without deviating from the spirit and scope of the invention.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A method for determining a presence of a damaging agent comprising:
receiving, from a sampling device, a pattern of a molecular sample;
analyzing, at a computing device comprising a virus and damaging agent machine-learning model and communicatively coupled to the sampling device, the received pattern of the molecular sample; and
based on the analyzing, identifying, using the virus and damaging agent machine-learning model, a particular damaging agent within the molecular sample, wherein the identifying is further based on the pattern exceeding an identification threshold.

2. The method of claim 1, wherein the pattern of the molecular sample includes at least one of time stamp information indicative of a time at which the molecular sample was captured, or geolocation information indicative of a global position at which the molecular sample was captured.

3. (canceled)

4. The method of claim 1, further comprising:
based on identifying the presence of the particular damaging agent, sending an alert to a user device, wherein the alert is indicative of the presence of the identified particular damaging agent, wherein the alert comprises time stamp information, geolocation information, or combinations thereof.

5.-6. (canceled)

7. The method of claim 1, the damaging agent is a virus, bacterium, parasite, protozoa, prion, or combinations thereof.

8.-9. (canceled)

10. The method of claim 4, further comprising:
based on receiving the alert indicative of the presence of the identified particular damaging agent, emitting, by a sanitizing agent emitter, a sanitizing agent capable of neutralizing at least the identified particular damaging agent.

11. The method of claim 1, wherein the pattern comprises a digital pattern.

12. The method of claim 1, wherein the pattern is formed responsive to a chemical reaction of a reagent with the damaging agent.

13. The method of claim 12, wherein the pattern is formed via a chromatographic immunoassay device.

14. The method of claim 1, identifying the particular damaging agent within the molecular sample is further based on using a virus and damaging agent detection machine-learning model of the computing device.

15.-16. (canceled)

17. A system comprising:
a sampling device configured to:
to generate a pattern of a molecular sample; and
a computing device comprising a virus and damaging agent detection machine-learning model and communicatively coupled to the sampling device, configured to:
receive, from the sampling device, the pattern of the molecular sample;
analyze the received pattern of the molecular sample; and
based on the analysis, identify a particular damaging agent within the molecular sample, wherein the identifying is further based on the pattern exceeding an identification threshold.

18. The system of claim 17, wherein the pattern comprises a digital pattern.

19. The system of claim 18, wherein the pattern is formed responsive to a chemical reaction of a reagent with the damaging agent.

20. The system of claim 19, wherein the pattern is formed via a chromatographic immunoassay device.

21. The system of claim 18, wherein the digital pattern of the molecular sample generated by the sampling device includes at least one of time stamp information indicative of a time at which the molecular sample was captured, or geolocation information indicative of a global position at which the molecular sample was captured.

22. (canceled)

23. The system of claim 17, wherein the computing device is further configured to:
based on identifying a presence of the particular damaging agent, sending an alert to a user device communicatively coupled to the sampling device, wherein the alert is indicative of the presence of the identified particular damaging agent, wherein the alert comprises time stamp information, geolocation information, or combinations thereof.

24. (canceled)

25. The system of claim 17, wherein the particular damaging agent identified by the computing device is a virus, bacterium, parasite, protozoa, prion, or combinations thereof.

26.-29. (canceled)

30. A method for training a virus and damaging agent detection machine-learning model used for detecting a damaging agent from a digital pattern of a molecular sample, the method comprising:
generating a three dimensional (3D) model of a particular damaging agent in a particular environment;
utilizing the 3D model to generate a plurality of output images, wherein the plurality of output images are captured at one or more of: different rotations of the 3D model, varying brightness levels, or varying magnification levels; and
training, using the plurality of output images, the virus and damaging agent detection machine-learning model to detect the damaging agent from the digital pattern of the molecular sample.

31.-33. (canceled)

34. The method of claim 30, wherein the virus and damaging agent detection machine-learning model comprises a plurality of feature detection models, including a first feature detection model and a second feature detection model.

35. The method of claim 34, wherein the first feature detection model is trained, using the plurality of output images, to detect a first feature of the particular damaging agent from the digital pattern of the molecular sample, and the second feature detection model is trained, using the plurality of output images, to detect a second feature of the particular damaging agent from the digital pattern of the molecular sample, wherein based on the first feature detection model detecting the first feature within the digital pattern and the second feature detection model detecting the second feature within the digital pattern, identifying the particular damaging agent within the digital pattern.

36. (canceled)

37. The method of claim 35, wherein:
detecting the first feature within the digital pattern is based at least on the digital pattern exceeding a first identification threshold,
the first identification threshold is associated with the first feature,
detecting the second feature within the digital pattern is based at least on the digital pattern exceeding a second identification threshold, and
the second identification threshold is associated with the second feature.

38.-45 (canceled)
